(19) United States
(12) Patent Application Publication
(10) Pub. No.: US 2003/0031718 A1
(43) Pub. Date: Feb. 13, 2003

(54) Ophthalmic compositions and use

(76) Inventors: Michelle Wong, Duluth, GA (US); Mary Sou, Alpharetta, GA (US); Shau-fong Yen, Atlanta, GA (US); Kasey Minick, Cary, NC (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/164,756

(22) Filed: Jun. 7, 2002

Related U.S. Application Data
(60) Provisional application No. 60/297,068, filed on Jun. 8, 2001.

(51) Int. Cl. A61K 31/4535; A61K 9/14

(52) U.S. Cl. 424/488; 514/320

(57) ABSTRACT

Disclosed are a once-a-day ophthalmic drug composition, comprising in particular ketotifen, comprising a polymer comprising chitosan and a carrier, and a method to treat an ocular allergy comprising administering a once-a-day ophthalmic ketotifen composition comprising chitosan to the eye of a mammal.

This invention is relates to ophthalmic compositions, e.g. gels, comprising in particular ketotifen or a pharmaceutically acceptable salt thereof for once-a-day administration. Ophthalmic compositions comprising ketotifen are known, e.g. from WO 01/07049, and commercially available, e.g. under the trade name Zaditen®. However, it has been found that these compositions typically have to be applied two to four times a day. It has been found that such repeated administration is not optimal in practice, because, inter alia, for optimal treatment the patient has to have the medicament always available and the patient is disturbed several times a day. Such multiple administration of a drug, in particular of an ophthalmic composition, leads generally to the problem of overdosing and/or underdosing. Overdosing may generate ocular irritation, whereas underdosing may lead to recurrence of the symptoms.

There is thus a need for methods and compositions that would allow once-a-day administration of ophthalmic drugs that previously required multiple administrations per day, e.g., ketotifen and pharmaceutically acceptable salts thereof. It has now been found that an ophthalmic drug such as ketotifen or its salts may be formulated for once-a-day administration which once-a-day administration provides therapeutic efficacy in the eye over about 24 hours and that such compositions are surprisingly well tolerated. Moreover the abovementioned once-a-day ophthalmic compositions produce a highly reliable and more reproducible clinical result in a patient treated therewith.

Therefore, in one aspect the present invention provides an ophthalmic composition suitable for topical once-a-day administration to the eye, comprising an ophthalmic drug such as ketotifen, a polymer comprising chitosan, and an ophthalmic carrier, hereinafter “composition(s) of the (present) invention”.

For example, the concentration of ketotifen hydrogen fumarate in the ophthalmic compositions of the invention is preferably from about 0.005-0.1%, more preferably from 0.01-0.05%, in particular from 0.02-0.04%, and especially 0.0345% by weight based on the total weight of the composition. Preferably the ketotifen hydrogen fumarate is in aqueous solution. If desired, however, the compositions of the present invention may be in the form of a suspension, e.g., may contain particles of ketotifen or salts thereof with particle diameters no greater than 90 micron.

As noted above, ketotifen may be in free base form or in pharmaceutically acceptable acid addition salt form, such as hydrochloride, hydrogen fumarate and the like. A preferred salt is the hydrogen fumarate.

Compositions of the present invention with moderate viscosity (e.g. between about 500 to 2000, or e.g between about 1000 and 2000 mPas at 20-25° C.) have been found to be convenient to apply but still have a good/excellent retention as indicated by a tears weight retention AUC of e.g. 100 to 400 microlitres/hour or mg/min-hr.

The excipients and amounts thereof may be chosen such that the viscosity of the compositions of the present invention will increase to the range indicated above upon instillation into the eye due to the change in temperature upon instillation (e.g., from storage temperatures, e.g. about 20° C., to about, e.g. 32-34° C., the temperature at the surface of the eye).

The compositions of the present invention may also comprise pharmaceutically acceptable excipients, which are ophthalmically compatible. For example, the excipients and/or compositions of the present invention should not significantly affect either the lacrimal system or the corneal tear film.

It has now been found that a polymer comprising chitosan is particularly suitable, especially under conditions specified herein, for inclusion in a once-a-day ophthalmic composition further containing an ophthalmic drug such as ketotifen. Although administrable in convenient drop form, such compositions achieve an advantageous retention time in the eye, with prolonged and sustained release of an ophthalmic drug in the eye. In addition, such formulations, especially those comprising ketotifen, provide surprisingly good ocular tolerability and excellent reproducibility of therapeutic efficacy.

A polymer comprising chitosan is preferably selected from the group consisting of chitosan, N,O-carboxyalkyl chitosan, such as N,O-carboxymethyl chitosan, O-carboxyalkyl chitosan such as O-carboxymethyl chitosan, and mixtures thereof, e.g., a mixture of N,O-carboxymethyl chitosan and O-carboxymethyl chitosan.

Chitosan as used in the present invention refers to a polymer consisting essentially of monomeric α(1→4)-D-glucosamine (A) linked units and of monomeric α(1→4)-N-acetyl-D--glucosamine (B) linked units which are scattered randomly in the molecule of the polymer, the numerical proportions of A and B being from about 60 to about 99% of A and about 1 to about 40% of B, the viscosity rating of the polymer being from about 3 to about 3000 centipoise (cps).

The polymers comprising chitosan useful in the compositions of the present invention are polymers with varying molecular weights, from about 10 thousand to about 10 million. The polymers have a corresponding viscosity rating which typically increases with molecular weight.

Chitosan in various forms is commercially available or can be prepared by deacetylation of chitin, e.g. as described in U.S. Pat. No. 3,953,068.

The chitosan is also characterized as to the proportion of N-acetyl-D-glycosamine units and D-glucosamine units, and such is expressed as the degree of deacetylation of the fully acetylated polymer chitin. Preferably the degree of deacetylation ranges from 70 to 90%, meaning that the proportion of N-acetyl-D-glucosamine units and D-glucosamine units in the chitosan is 10 to 30% of N-acetyl-D-glucosamine units and 70 to 90% of D-glucosamine units. The degree of deacetylation (free amine) can be determined by dissolving a sample in dilute hydrochloric acid, and back titrating the excess acid with dilute sodium hydroxide.

In general, the viscosity of the chitosan-containing compositions of the invention increases with increasing pH due to, inter alia, the reversible interconversion of the ammonium salt form of the D-glucosamine units within the polymer to the free amine form thereof, thus resulting in a more water insoluble form of the polymer carrier. The pH of chitosan-containing solutions can be adjusted according to methods well known in the art, e.g. with an acid such as hydrochloric acid to decrease the pH, or with a base such as sodium hydroxide to raise the pH. The pH can be adjusted to obtain a viscosity suitable for administration in drop form. The highest possible pH at which the composition can be administered in drop form is selected so that any irritation to the eye is minimized.

Preferred pH of a composition of the present invention ranges from about 4.0 to about 8.0, advantageously about 5.5 to about 7.5, and preferably from about 6.0 to about 7.0.

Other factors affecting viscosity include the properties of the chitosan used (viscosity rating, molecular weight, proportion of acetylated to non-acetylated D-glucosamine units), and the concentration of the chitosan.

Typical concentrations of chitosan being present in a composition of the present invention ranges from about 0.05% to about 10% by weight of the total composition, preferably from about 0.1 to about 5%, advantageously from about 0.5 to about 4%, and in particular from about 1 to about 3%.

The viscosity of the chitosan-containing compositions of the invention which are administrable in drop form is between about 10 cps and about 100,000 cps at a shear rate of 1 sec-1, preferably between about 100 to 80,000 cps, and advantageously between about 500 and 10,000 cps as measured using the Bohlin CS Rheometer (Plate-Plate Geometry). The viscosity of the present formulations remains substantially constant over a wide range of shear rates.

O-carboxyalkyl chitosan, as used herein, is defined according to formula (I),

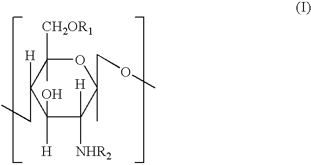

where R₁ is H about 0 to 82% of the time and carboxyalkyl about 18-100% of the time, and where R₂ is H about 70 to 90% of the time and acetyl about 30 to 10% of the time. The O at the 3-position may be substituted with R₁ some of the time, but the species shown is the predominant species.

As used herein, carboxyalkyl preferably refers to carboxymethyl.

Unless indicated differently, alkyl refers throughout this invention to an alkyl group having up to and including 10, more preferably 6 and even more preferably 3 C-atoms, and is either a linear or a branched alkyl group.

Examples for alkyl are methyl, ethyl, propyl, butyl, iso-propyl, t-butyl, neo-pentyl, octyl or decyl.

The O-carboxyalkyl chitosan, in particular the O-carboxymethyl chitosan molecular weight may range from about 1000 Daltons to about 5,000,000 Daltons, depending on the intended use of the final product. The compositions of the present invention may have a viscosity of about 1 to 200,000 centipoise at 25° C., depending again on the intended use of the product. A preferred viscosity range for an ophthalmic product which delivers an agent to the ocular environment is about 50 to 100,000 centipoise. A preferred viscosity range is about 50 to 5000 centipoise.

The compositions of the present invention include about 0.01 to about 25 weight percent O-carboxyalkyl chitosan, in particular O-carboxymethyl chitosan. More preferably, the ophthalmic product includes about 0.1 to about 10 weight percent and especially about 0.3 to about 5 weight percent O-carboxyalkyl chitosan.

O-carboxyalkyl chitosan may be formulated at a pH of about 4 or higher without precipitation out of solution. Thus, preferably, the compositions have a pH of about 4 or higher, more preferably about 4.5 or higher. In a preferred embodiment, the compositions of the present invention have a pH of about 4 to 9. In another preferred embodiment, the compositions of the present invention have a pH of about 4 to 8. In a further preferred embodiment the compositions of the present invention have a pH of about 5.0 to 7.8.

N,O-carboxyalkyl chitosans are derivatives of chitosan formed by carboxyalkylation of chitosan. The carboxyalkyl groups of N,O-carboxyalkyl chitosan are located at the primary amino group on the D-glycosamine group and at the hydroxyl groups. N,O-carboxymethyl chitosan is water soluble and may be formed by carboxymethylation of chitosan. For example, the preparation of N,O-carboxymethyl chitosan is disclosed in U.S. Pat. No. 4,619,995.

A preferred N,O-carboxyalkyl chitosan is N,O-carboxymethyl chitosan.

The compositions of the present invention include about 0.1 to about 25 weight percent N,O-carboxyalkyl chitosan, in particular N,O-carboxymethyl chitosan. More preferably, such composition includes about 0.1 to about 5 weight percent and especially about 0.25 to about 5 weight percent N,O-carboxyalkyl chitosan.

N,O-carboxyalkyl chitosan may be formulated at a pH of about 5.5 or higher without significant precipitation out of solution. Thus, preferably, the compositions of the present invention have a pH of about 5.5 or higher, more preferably about 6.0 or higher. In a preferred embodiment, the compositions of the present invention have a pH of about 6 to 9

The N,O-carboxyalkyl chitosan, in particular N,O-carboxymethyl chitosan molecular weight may range from about 1000 Daltons to about 5,000,000 Daltons, depending on the intended use of the final product. The compositions of the present invention may have a viscosity of about 1 to 200,000 centipoise at 25° C., depending again on the intended use of the product. A preferred viscosity range for the compositions of the present invention which deliver an agent to the ocular environment is about 30 to 100,000 centipoise.

The compositions of the present invention may further comprise a tonicity enhancing agent.

Tonicity enhancing agents are, for example, ionic compounds, such as alkali metal or alkaline earth metal halides, such as, for example, CaCl2, KBr, KCl, LiCl, Nal, NaBr or NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol, preferred from 100 to 400 mOsmol, more preferred from 200 to 400 mOsmol and even more preferred from 280 to 350 mOsmol.

Surprisingly, it has been found in the compositions of the invention that non-ionic tonicity enhancers such as mannitol and the chitosan-comprising polymers such as those described hereinbefore appear to act synergistically with respect to the excellent retention, stability and tolerability in compositions of the invention. For example, very little degradation is observed when a composition of the present invention is heat sterilized.

For the adjustment of the pH, preferably to a physiological pH, buffers may especially be useful. Examples of buffer substances are acetate, ascorbate, borate, hydrogen carbonate/carbonate, citrate, gluconate, lactate, phosphate, propionate and TRIS (tromethamine) buffers. Tromethamine and borate buffer are preferred buffers. The amount of buffer substance added is, typically, that necessary to ensure and maintain a physiologically tolerable pH range. The pH range is generally in the range of from 4 to 9, preferably from 4.5 to 8.5 and more preferably from 5.0 to 8.2.

The compositions of the present invention may further comprise a preservative, e.g. on storage or to inhibit microbial growth after opening a closed container holding such a composition and exposing such a composition to the air.

A preservative may typically be selected from a quaternary ammonium compound such as benzalkonium chloride, benzoxonium chloride or the like. Benzalkonium chloride is better described as: N-benzyl-N-(C8-C18alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Polyquat®, Germal®II or sorbic acid. Preferred preservatives are quaternary ammonium compounds, in particular benzalkonium chloride, sodium perborate and Polyquat. Where appropriate, a sufficient amount of preservative is added to the compositions of the present invention to ensure protection against secondary contaminations during use caused by bacteria and fungi.

A composition of the present invention may additionally require the presence of a solubilizer, in particular if the active or the inactive ingredients tend to form a suspension or an emulsion. A solubilizer suitable for compositions of the invention is for example selected from the group consisting of tyloxapol, fatty acid glycerol polyethylene glycol esters, fatty acid polyethylene glycol esters, polyethylene glycols, glycerol ethers, a cyclodextrin (for example α-, β- or γ-cyclodextrin, e.g. alkylated, hydroxyalkylated, carboxyalkylated or alkyloxycarbonyl-alkylated derivatives, or mono- or diglycosyl-α-, β- or γ-cyclodextrin, mono- or dimaltosyl-α-, β- or γ-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH 40®. Reaction products of castor oil and ethylene oxide appear to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is typically from 0.1 to 5000 times the concentration of the active ingredient.

Further excipients may be comprised in a composition of the invention, which may in particular function as a combined stabilizer/solubilizer. Such a combined additional stabilizer/solubilizer is for example a cyclodextrin. A preferred cyclodextrin is in particular selected from the group of α-cyclodextrin, β-cyclodextrin, γ-cyclodextrin, hydroxypropyl-α-cyclodextrin, hydroxypropyl-β-cyclodextrin, dimethyl-α-cyclodextrin and dimethyl-β-cyclodextrin. The amount is generally in the range of from approximately 0.01 to approximately 90% by weight, more preferably in the range of from 0.1-20% by weight.

A composition of the invention may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. Other excipients that may be used if desired are listed below but they are not intended to limit in any way the scope of the possible excipients. They are especially complexing agents, such as disodium-EDTA, EDTA, phosphonic acids such as those disclosed in U.S. Pat. No. 5,725,887, antioxidants, such as ascorbic acid, acetylcysteine, cysteine, sodium hydrogen sulfite, butyl-hydroxyanisole, butyl-hydroxytoluene or alpha-tocopherol acetate; stabilizers, such thiourea, thiosorbitol, sodium dioctyl sulfosuccinate or monothioglycerol; or other excipients, such as, for example, lauric acid sorbitol ester, triethanol amine oleate or palmitic acid ester. Preferred excipients are complexing agents, such as disodium-EDTA. The amount and type of excipient added is in accordance with the particular requirements and is generally in the range of from approximately 0.0001 to approximately 90% by weight.

A composition of the present invention is preferably in form of clear solution or gel, e.g. clear gel, and may, optionally, be clarified by filtration, e.g., through a sterile filter.

A composition of the present invention is stable, as indicated by conventional tests, e.g under stressed conditions, such as 15 h at 80° C. or 40° C. at 15% relative humidity. A composition of the present invention is typically stable over 2 even 3 years showing less than 5% degradation of the active (e.g. ketotifen) at 20° C. to 30° C.

A composition of the present invention may be packaged in conventional manner. A composition of the present invention may be stored in single or multiple unit dosage form, e.g. closed bottles, tubes or other containers made from glass or plastic (polyethylene, polyethylene terephthalate or polypropylene). For example bottles may contain about 1 to 5 ml of the compositions of the present invention. The container may be fitted with a dropper to facilitate administration.

It will be appreciated that although the excipients have been described above by reference to a particular function any particular excipient may have alternative or multiple functions, e.g chitosan may act as both a preservative and viscosity enhancing agent.

Ophthalmic carriers are comprised in a composition of the present invention as well. Such carriers are typically adapted for topical administration, and are for example water, mixtures of water and water-miscible solvents, such as C1- to C7-alkanols, vegetable oils or mineral oils comprising from 0.5 to 5% by weight hydroxyethylcellulose, ethyl oleate, carboxymethyl-cellulose, polyvinyl-pyrrolidone and other non-toxic water-soluble polymers for ophthalmic uses, such as, for example, cellulose derivatives, such as methylcellulose, alkali metal salts of carboxy-methylcellulose, hydroxymethylcellulose, hydroxyethylcellulose, methylhydroxypropyl-cellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenan, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers.

Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof. A highly preferred carrier is water. The concentration of the carrier is, for example, from 1 to 100000 times the concentration of the active ingredient.

A composition of the present invention further comprising ketotifen or a pharmaceutically acceptable salt thereof is in particular useful for the temporary prevention of itching of the eye due to allergic conjunctivitis and may be used for the treatment and/or prevention of allergic conditions, selected from allergic conjunctivitis, seasonal allergic conjunctivitis, and allergic conditions treatable by ketotifen therapy, as indicated e.g in standard animal trials and clinical trials. A ketotifen composition of the present invention is effective in both animal, e.g. mammal and human.

The compostions of the present invention comprise an active ingredient at a concentration so that an effective amount thereof is contained in a drop, wherein said drop amounts about 10-100 μl (microliters), preferably about 20-70 μl, and especially about 25-50 μl.

The following examples are presented for illustrative purposes. Said examples are relating to a specific ophthalmic drug, ketotifen (free base) and ketotifen hydrogen fumarate but are not intended to limit the scope of the invention. A 0.0345% solution of ketotifen hydrogen fumarate provides a solution of 0.025% ketotifen as free base, e.g., as in the commercial product Zaditen® 0.025%. It will be appreciated by the skilled man in the art that the present invention is virtually applicable to any ophthalmic drug and especially also to any antiallergic ophthalmic drug known and described in the art.

It shall be further appreciated that the present invention also relates to any aspect of any dependant and/or independent claim as disclosed hereinafter.

In particular, the invention relates to a method for treating and/or preventing an ocular allergic condition, comprising a single topical administration per day of an effective amount of a once-a-day antiallergic drug composition, e.g. ketotifen, to the eye of a patient in need thereof, which composition comprises an anti-allergic drug, e.g. ketotifen, a polymer comprising chitosan, and an ophthalmic carrier.

In a further aspect the present invention provides: a once-a-day ophthalmic composition comprising ketotifen as defined above and below for use in the treatment of allergic conjunctivitis and in particular of treatment and prevention of seasonal allergic conjunctivitis, or a condition treatable by ketotifen therapy; a method for treating allergic conjunctivitis and in particular for treating and preventing seasonal allergic conjunctivitis or a condition treatable by ketotifen therapy comprising a single administration per day of a composition of the present invention to the eye of a patient in need thereof, which composition comprises an antiallergic drug such as ketotifen; the use of a composition of the present invention, containing an antiallergic drug, in particular ketotifen, in the preparation of a medicament for the treatment of allergic conjunctivitis and in particular of treatment and prevention of seasonal allergic conjunctivitis or a condition treatable by ketotifen therapy.

Clinics

A clinical trial may be effected comparing, according to a standard score-card, the good efficacy and tolerability (lack of irritation) of about 50 microliters of compositions of the present invention containing 0.025% of ketotifen topically administered once a day by instillation onto the ocular surface, e.g. to the inside lower lid, to groups of, e.g 10, healthy volunteers, or patients suffering from allergic conjunctivitis, with the commercially available product Zaditen® 0.025% ketotifen eye drops administered 3 (2-4) times a day. The trial lasts typically 8 days.

Tolerance

The eyeballs of the subjects are examined visually to determine the retention of the compositions of the present invention adjacent to the eye, the good tolerability (e.g by lack of significant irritation or reddening) and effect against conjunctivitis, e.g fast onset of action and long duration of action.

Bioavailability:

Additionally the bioavailability of the compositions of the present invention in the above trials as determined by absorption in the conjunctiva are compared to Zaditen® 0.025% administered 3 times a day.

The bioavailibity of an addressed once-a-day ophthalmic composition is assessed with the pharmacokinetic assay described herein:

Fifty microliters of the ophthalmic formulation is instilled onto the upper part of the conjunctiva of rabbits. Tears, bulbar conjunctiva, cornea and sciera are sampled after either 5, 15, 30 minutes, or, 1, 8, 16, or 20 hours. Samples are extracted for ketotifen determination related to the wet weight amount of tissue or tears.

Ketotifen is determined with a liquid chromatography linked to mass spectrometry validated method.

Retention Tests:

Additional in this test, retention of ketotifen onto the ocular surface is observed after instillation of a 50 microliter single dose of formulations of the present invention as indicated by sampling of tears versus time and determining amounts of ketotifen in such tears.

The retention of an addressed once-a-day ophthalmic composition is assessed with retention tests described herein:

The albino rabbits are held gently without any constraint box. The upper eyelid of one eye is then carefully pulled away from the eyeball and 50 μl (microliter) of the test substance is instilled on the superior part of the bulbar conjunctiva using a gauged automatic pipette. The eyelid is gently closed for one second. After either 5 min, 1 hour, 4 hours, 8 hours, 16 hours or 24 hours a weighed Schirmer's test strip is maintained in the cul-de-sac between the inferior lid and the temporal part of the eyeball for one minute. The absorbed tears are immediately weighed and kept frozen up to the time when the analytical test of ketotifen content is carried out.

The analytical method is based on liquid chromatography/negative ion chemical ionization mass spectrometry.

To achieve high precision and low variability and to facilitate peak identification, a deuterated analogue of Ketotifen is prepared as internal standard. By an acid-catalysed exchange reaction, the deuterated compound is readily available starting from the intact target molecule.

Deuteration of Ketotifen for Internal Standard

Around 100 mg (milligrams) of ketotifen salt is dissolved in D2O. The resulting solution is treated with 40% NaOD in D2O (pH 12-14) in order to convert ketotifen salt to its free base form. The solid is collected by suction filtration, washed with D2O, dried in vacuo at ambient temperature and then dissolved in 20 drops D2O and 60 drops 37% DCI/D2O. The resulting mixture is heated at 110° C. for 6 hours, cooled down at ambient temperature and finally treed with 40% NaOD in D2O (pH 13-14). A green residue, which stuck to the vial, and a free white residue are formed. The white residue is collected by suction, washed with D2O, dried under high vacuum at ambient temperature and reconstituted in 4 ml of acetonitrile. The resulting stock solution is identified as deuterated ketotifen solution by LC-MS. Deuterium-hydrogen back-exchange is investigated by adding deuterated ketotifen to tears. No back-exchange is detectable over a period of 24 hours.

Sample Processing

A known concentration of internal standard is added to the Schirmer test strips. All are then extracted in one ml of a mixture of acetonitrile and water (50:50) on a rotary shaker. After centrifugation, the supernatant is collected and analysed by LC-MS.

The amount of ketotifen collected on each Schirmer test strip is then calculated from the integrated peak areas against a dilution series of test compound. The resulting value is finally corrected by taking into account the extraction yield (determined with the internal standard) and the weight of tears collected on each strip.

### EXAMPLE 1

Active Ingredient: Ketotifen hydrogen fumarate Concentration: 0.034% (0.025% base)

| Ingredient | Concentration¹ |
|---|---|
| Ketotifen hydrogen fumarate | 0.0345% |
| Chitosan (Protasan G113) | 0.5% |
| Benzalkonium Chloride* | 0.005% |
| Mannitol | 4.5% |
| Sodium Bicarbonate^{‡} | qs to pH 6.0 |
| Water for Injection | qs to 100% |

Sterilization method: sterile filtration with a 0.2 micron filter Benzalkonium chloride is made into a 10% solution and tested, then adjusted accordingly when added to the formulation. Sodium Bicarbonate is made into a 5% solution and added to adjust pH.

### EXAMPLE 2

Active Ingredient: Ketotifen hydrogenfumarate Concentration: 0.034% (0.025% base)

| Ingredient | Concentration¹ |
|---|---|
| Ketotifen hydrogen fumarate | 0.0345% |
| N,O-carboxymethyl Chitosan (High Viscosity) | 1.0% |
| Benzalkonium Chloride* | 0.005% |
| Mannitol | 4.3% |
| Edetate Disodium | 0.05% |
| 1.0N Hydrochloric Acid | qs to pH 6.0 |
| Water for Injection | qs to 100% |

Sterilization method: steam sterilization; ketotifen added aseptically by filtration through a 0.2 micron filter Benzalkonium chloride is made into a 10% solution and tested, then adjusted accordingly when added to the formulation.

### EXAMPLE 3:

Active Ingredient: Ketotifen hydrogen fumarate Concentration: 0.034% (0.025% base)

| Ingredient | Concentration¹ |
|---|---|
| Ketotifen hydrogen fumarate | 0.0345% |
| N,O-carboxymethyl Chitosan (Low Viscosity) | 1.0% |
| Benzalkonium Chloride* | 0.005% |
| Mannitol | 4.3% |
| Edetate Disodium | 0.05% |
| 1.0N Hydrochloric Acid | qs to pH 6.0 |
| Water for Injection | qs to 100% |

Sterilization method: steam sterilization; ketotifen added aseptically by filtration through a 0.2 micron filter. Benzalkonium chloride is made into a 10% solution and tested, then adjusted accordingly when added to the formulation.

### EXAMPLE 4

Active Ingredient: Ketotifen hydrogen fumarate Concentration: 0.034% (0.025% base)

| Ingredient | Concentration¹ |
|---|---|
| Ketotifen hydrogen fumarate | 0.0345% |
| N,O-carboxymethyl Chitosan (Low Viscosity) | 0.3% |
| Benzalkonium Chloride* | 0.005% |
| Mannitol | 4.3% |
| Edetate Disodium | 0.05% |
| 1.0N Hydrochloric Acid | qs to pH 6.0 |
| Water for Injection | qs to 100% |

Sterilization method: steam sterilization; ketotifen added aseptically by filtration through a 0.2 micron filter. Benzalkonium chloride is made into a 10% solution and tested, then adjusted accordingly when added to the formulation.

| | A | B | C | D |
|---|---|---|---|---|
| | % w/v | % w/v | % w/v | % w/v |
| Composition | | | | |
| Ketotifen Hydrogen Fumarate | 0.0345 | 0.0345 | 0.0345 | 0.0345 |
| O-carboxy methyl Chitosan (*) | 1.0 | 1.0 | 5.0 | 5.0 |
| | (P38OCMSAN4) | (P38OCMSAN7) | (P38OCMSAN4) | (P38OCMSAN7) |
| Benzalkonium chloride | 0.005 | 0.005 | 0.005 | 0.005 |
| 1.0 N HCl qs | qs to pH-6 | qs to pH-6 | qs to pH-6 | qs to pH-6 |
| Mannitol | 4.95 | 4.95 | 4.5 | 4.5 |
| EDTA | 0.05 | 0.05 | 0.05 | 0.05 |
| Water for injection ad | 100 ml | 100 ml | 100 ml | 100 ml |
| *product provided by Chitogenics: | | | | |
| P38OCMSAN4 and P38OCMSAN7 | | | | |
| Appearance, colour | Clear, colorless | Clear, colorless | Clear, colorless | Clear, colorless |
| | solution | solution | solution | solution |
| Initial value | | | | |
| pH | 6.18 | 6.12 | 5.99 | 5.98 |
| Osmolality (mOsm) | 298 | 292 | 290 | 288 |
| Stress test 15 h 80° C. (in 5 ml PP- | | | | |
| bottles) Loss of ketotifen content in | | | | |
| % | | | | |
| pH | 4.4 | 4.4 | 6.0 | 3.6 |
| Osmolality (mOsm) | 6.18 | 6.09 | 5.98 | 5.97 |
| Aspect, color | 289 | 292 | 295 | 290 |
| | Clear colorless | Clear colorless | Clear colorless | Clear colorless |
| | solution | solution | solution | solution |
| Preservative Effectiveness Test | Passes criteria A | Passes criteria A | Passes criteria A | Passes criteria A |
| | per Ph. Eur. | per Ph. Eur. | per Ph. Eur. | per Ph. Eur. |

The compositions of the present invention are stable, as indicated by conventional tests, e.g. under stressed conditions, such as 15 hr at 80° C. or 40° C. at 15% relative humidity. The compositions of the present invention are stable over 2, even 3 years showing less than 10% degradation of ketotifen at 20 to 30° C.

1. An ophthalmic composition for topical once-a-day administration to the eye, comprising an ophthalmic drug, a polymer comprising chitosan and an ophthalmic carrier.

2. The composition of claim 1, wherein said polymer comprising chitosan is selected from the group consisting of chitosan, an N,O-carboxyalkyl chitosan, an O-carboxyalkyl chitosan, and mixtures thereof.

3. The composition of claim 1, wherein said drug is ketotifen or a pharmaceutically acceptable acid addition form thereof.

4. The composition of claim 3, wherein said drug is present in said composition at a concentration between about 0.005 and about 0.1%.

5. The composition of claim 1, wherein said polymer comprising chitosan is chitosan consisting essentially of monomeric β(1→4)-D-glucosamine (A) linked units and of monomeric β(1→4)-N-acetyl-D-glucosamine (B) linked units which are scattered randomly in the molecule of the polymer, wherein the proportions of A and B are from about 60 to about 99% of A and about 1 to about 40% of B, wherein the viscosity of the polymer is from about 3 to about 3000 cps.

6. The composition of claim 1, wherein the concentration of the polymer comprising chitosan ranges from about 0.05% to about 10% by weight of the total composition.

7. The composition of claim 1, wherein said polymer is N,O-carboxyalkyl chitosan.

8. The composition of claim 1, wherein polymer is O-carboxyalkyl chitosan.

9. The composition of claim 1, wherein said carrier is water.

10. The composition of claim 3, wherein said drug is ketotifen hydrochloride or ketotifen hydrogen fumarate.

11. The composition of claim 1 which comprises ketotifen hydrogen fumarate, a polymer comprising chitosan, a preservative, a tonicity enhancer, and an ophthalmic carrier.

12. The composition of claim 11, wherein said polymer is N,O-carboxyalkyl chitosan.

13. The composition of claim 11, wherein said polymer is O-carboxyalkyl chitosan.

14. The composition of claim 11, wherein said polymer is chitosan consisting essentially of monomeric β(1→4)-D-glucosamine (A) linked units and of monomeric β(1→4)-N-acetyl-D-glucosamine (B) linked units which are scattered randomly in the molecule of the polymer, wherein the proportions of A and B are from about 60 to about 99% of A and about 1 to about 40% of B, wherein the viscosity of the polymer is from about 3 to about 3000 cps.

15. A method for treating or preventing an ocular allergic condition in a subject in need of such treatment, comprising a once daily topical administration to the eye of said subject of an effective amount of an ophthalmic ketotifen composition, wherein the composition comprises ketotifen or a pharmaceutically acceptable salt thereof, a polymer comprising chitosan, and an ophthalmic carrier.

16. The method of claim 15, wherein said allergic condition is selected from allergic conjunctivitis, seasonal allergic conjunctivitis, and allergic condition treatable by ketotifen therapy.

17. The method of claim 15, wherein said polymer comprising chitosan is selected from the group consisting of chitosan, N,O-carboxyalkyl chitosan, O-carboxyalkyl chitosan, and mixtures of N,O-carboxyalkyl chitosan and O-carboxyalkyl chitosan.

18. The method of claim 15, wherein said effective amount comprises about 10 to about 100 μl of said ophthalmic ketotifen composition.

19. The method of claim 15, wherein the concentration of said ketotifen or said pharmaceutically acceptable salt thereof in said ophthalmic ketotifen composition is between about 0.005% and about 0.1%.

20. The method of claim 15, wherein said patient is a mammal.

21. The composition of claim 2 wherein said N,O-carboxyalkyl chitosan is N,O-carboxymethyl chitosan and said O-carboxyalkyl chitosan is O-carboxymethyl chitosan.

22. The composition of claim 4, wherein the drug is present in a concentration between about 0.02 and about 0.04%.

23. The composition of claim 22, wherein the drug is present in a concentration of about 0.0345%.

24. The composition of claim 6, wherein the concentration of the polymer comprising chitosan ranges from about 0.01% to about 4%.

25. The composition of claim 7, wherein said polymer is N,O-carboxymethyl chitosan.

26. The composition of claim 8, wherein said polymer is O-carboxymethyl chitosan.

27. The composition of claim 3, wherein said drug is ketotifen hydrogen fumarate.

28. The composition of claim 11, wherein said tonicity enhancer is mannitol and said ophthalmic carrier is water.

29. The composition of claim 12, wherein said polymer is N,O-carboxymethyl chitosan.

30. The composition of claim 13, wherein said polymer is O-carboxymethyl chitosan.

31. The method of claim 17 wherein wherein said N,O-carboxyalkyl chitosan is N,O-carboxymethyl chitosan and said O-carboxyalkyl chitosan is O-carboxymethyl chitosan.

32. The method of claim 18, wherein said effective amount comprises between about 20 and about 70 μl.

33. The method of claim 33, wherein said effective amount comprises between about 25 and about 50 μl.

34. The method of claim 19, wherein said composition comprises ketotifen hydrogen fumarate and the concentration of said ketotifen hydrogen fumarate in said ophthalmic ketotifen composition is about 0.0345%.
